(19) United States
(12) Patent Application Publication
(10) Pub. No.: US 2004/0153040 A1
(43) Pub. Date: Aug. 5, 2004

(54) Multi-layer synthetic dressing with cooling characteristics

(76) Inventors: Lucie Martineau, Kettleby (CA); Pang N. Shek, Toronto (CA)

Correspondence Address: D.W. EGGINS, 18 DOWNSVIEW DRIVE, BARRIE, ON, L4M 4P8, CA

(21) Appl. No.: 10/428,775

(22) Filed: May 5, 2003

Related U.S. Application Data
(62) Division of application No. 10/358,165, filed on Feb. 5, 2003.

(51) Int. Cl. A61F 13/00

(52) U.S. Cl. 604/304

(57) ABSTRACT

A multi-layered polyurethane foam dressing with cooling properties for use in body cavities, on damaged tissues, particularly burns, or for cosmetic use. The dressing includes: ¹⁾ an optional outer layer of either a hydrogel formulated from a polyurethane or an adhesive elastomeric material; ²⁾ a hydrophilic polyurethane foam layer; ³⁾ a non-adherent surface-contacting cooling layer of a polyurethane hydrogel; and, ⁴⁾ an optional protective cover-sheet. An interposed liquid transfer control may be used at a layer interface. The dressing can be in various shapes and sizes (e.g., cylindrical, oval, etc. or flat sheets). A secondary wrapping dressing may be applied to secure the dressing. The contact surface may be channeled to enhance fluid distribution.

### CROSS-REFERENCE TO RELATED APPLICATIONS

This application is a division of application Ser. No. 10/358,165, filed Feb. 5, 2003

### STATEMENT REGARDING FEDERALLY SPONSORED RESEARCH OR DEVELOPMENT

Not Applicable (N/A)

### REFERENCE TO A SEQUENCE LISTING, A TABLE, OR A COMPUTER PROGRAM LISTING

(N/A)

### COMPACT DISC APPENDIX

(N/A)

### BACKGROUND OF THE INVENTION

This invention is directed to an absorbent, synthetic pad particularly suited for use as a dressing. More particularly, one embodiment of the present invention relates to a polyurethane-based dressing made up of at least two layers, the surface-contacting layer possessing particular cooling characteristics. The dressing comprises one layer of hydrophilic polyurethane foam, preferably HYPOL polyurethane, and at least one surface-contacting layer of hydrogel, preferably HYPOL hydrogel. The hydrophilic layer functions as a reservoir capable of absorbing excess exudates, while the hydrogel layer acts as a minimally adherent surface that maintains the wound bed adequately moist to promote optimal wound healing, and exerts a soothing, cooling effect. The latter hydrogel layer may require to be perforated for purposes of fluid passage to the adjoining layer.

Wound care constitutes an important aspect of wound healing, and can play an integral role in post-operative treatment. Wound care regimens have changed considerably over the last three decades, through a better understanding of the physiological mechanisms underlying wound healing. This knowledge has led to the development of many types of wound dressings, all claimed to be useful for healing wounds.

While many commercially available wound dressings are recommended as low-adherent wound dressings, they have a tendency to dry. Unless these dressings are changed very frequently, they become incorporated into the newly formed granulation tissue, thus causing undesirable damage to a healing wound site upon their removal. The absorbency capacity of many recommended dressings is minimal, thus limiting their use primarily to wounds with low exudates.

It is herein postulated that an ideal wound dressing should be highly absorbent to wound exudates; minimally adherent to the wound bed to reduce the risk of re-injury upon removal of the dressing; and, exert a soothing and/or cooling effect upon application to the wound, especially a burn wound. There presently appears to be no known commercially available dressing that possesses all of the above desirable characteristics, nor such is taught in the prior art. Thus, the wound dressing of the present invention was created to overcome the shortcomings of conventional wound dressings.

A dressing with these characteristics may also be used for cosmetic, dental or therapeutic applications.

A number of prior art patents are listed, and discussed in detail in the attached Information Disclosure Statement, which forms a part of the present disclosure, and is incorporated herein by way of reference.

Hydrogels are important wound care products, with a unique ability to maintain the wound bed moist, and to cool the surface on which they are applied. However, a distinct disadvantage of commercially available hydrogel wound dressings is that they typically contain a high water content (>90%). This characteristic significantly restricts their absorbency capacity, and a secondary dressing is usually required to absorb any excess wound exudate.

Commercially available polyurethane wound dressings are therefore important wound care products, especially since they can absorb moderate to high volumes of wound exudate. The patent literature is also replete with references to multi-layer polyurethane wound dressings (preferably made of HYPOL polyurethane) in which therapeutic substances may be incorporated. However, there is no disclosure in the prior art that the layers of the multi-layered polyurethane dressing comprise a polyurethane layer and a hydrogel layer directly in contact with the skin or the wound, as disclosed in the wound dressing of the present invention.

Unlike certain prior art discussed in the accompanying Information Disclosure Statement, the multi-layer dressing according to the present invention has no adhesive layer on the contact face of the dressing, but has a minimally adherent is HYPOL hydrogel or any other suitable hydrogel as the surface-contacting layer. Another of the advantageous features of the present wound dressing is that the surface-contacting HYPOL hydrogel layer has a high water content to promote cooling upon application to a surface of a host such as a vertebrate host, and to reduce adhesion of the dressing to the wound surface. A further novel characteristic is that the layers of the subject dressing comprise HYPOL of different physical/chemical characteristics.

### SUMMARY OF THE INVENTION

This invention provides a dressing, comprising an optional outer layer of either hydrogel or elastomeric material; a hydrophilic polymer foam layer; a surface-contacting layer comprised of hydrogel; and, an optional protective release sheet.

The optional release sheet is divided into two separable, overlapping portions. The ready-to-use dressings are sealed in pouches that are preferably impermeable to water vapor, and the packaged unit can be sterilized (e.g., by gamma-irradiation).

More specifically, the present invention comprises a dressing having at least two primary layers. The first primary layer is preferably composed of HYPOL polyurethane foam pre-polymer, or another similar hydrophilic polyurethane pre-polymer (hereinafter, hydrophilic polyurethane pre-polymers will be referred to generically as HYPOL), which serves as a fluid-retention reservoir. The second primary layer is a minimally adherent surface-contacting layer, and preferably composed of HYPOL hydrogel or any other suitable hydrogel. The surface-contacting hydrogel layer may require to be discontinuous or perforated to enhance fluid absorption.

In another embodiment, the first primary HYPOL fluid-reservoir foam layer can be sandwiched between two surface-contacting or “face” layers of HYPOL hydrogel (or any other suitable hydrogel), for use as a packing material in deep wounds or body cavities. Each of the layers of HYPOL (i.e., surface-contacting and HYPOL layers) in the dressing may have different physical/chemical characteristics.

The formulation of the different HYPOL layers, which defines their physico-chemical characteristics, is determined by the functional role of each layer. For example, the surface-contacting HYPOL hydrogel layer has a higher water content to promote cooling upon its being applied to a surface of a host such as a vertebrate host, and to reduce adhesion of the dressing to the wound surface. The hydrophilic foam layer has a physico-chemical composition, made up of HYPOL and possibly other blending agents, which promote the absorption of fluid and may potentially favor the release of entrapped therapeutic agents. A HYPOL layer that serves the primary function of removing and retaining wound exudate fluid requires a physico-chemical characteristic that promotes moisture take-up and retention.

It will be understood that the positioning, in mutually adhering relation, of two surfaces having markedly different hydration levels, such as the hydrophilic HYPOL layer and the surface-cooling hydrogel layer in the subject dressing, may cause a transfer of moisture from one layer to the other. This potential moisture transfer can be addressed by modifying appropriately the physico-chemical composition of either layer.

In another embodiment, the initial moisture level of the hydrophilic HYPOL fluid-reservoir foam layer can be altered to affect the cooling capacity of the surface-contacting HYPOL hydrogel layer.

In yet another preferred embodiment, a self-regulating, flow-sensitive polymeric or synthetic membrane is placed between the hydrophilic HYPOL layer and the hydrogel layer, with the intent that the membrane prevents passive moisture transfer from the hydrogel layer to the HYPOL layer, thereby prolonging the cooling properties of the hydrogel layer. The presence of a moderate to high flow of exudate triggers the physical modification of the membrane to facilitate moisture transfer to the fluid-retaining HYPOL foam layer.

In a preferred embodiment of the dressing device of the present invention, the use of HYPOL polyurethane pre-polymers, with different physico-chemical characteristics, enables the use of an existing known chemical process intrinsic to polyurethane polymers to cure the two layers together, thereby avoiding but not necessarily excluding in future developments the use of existing known processes (e.g., heat sealing, welding by radio frequency welding, ultrasonic welding, adhesives) to laminate such layers together.

It is an object of this invention to provide a method for treating external wounds using a polyurethane foam dressing (preferably HYPOL polyurethane) comprised of at least two layers.

Yet another object of the invention is to provide a method for treating external wounds, using a multi-layer polyurethane foam dressing (preferably HYPOL polyurethane) that has a minimally adherent surface-contacting layer that will provide immediate cooling to the surface to which it is applied, and has a fluid-retaining layer that will thereafter absorb excess exudates.

The safe time, over which a dressing of the present invention may remain in a body cavity or wound, will vary depending on the type of wound, the immediate condition of the site, and the form of any other treatment which may be given to the wound.

The dressing of the present invention is particularly useful as a wound dressing applied to external injuries such as abrasions, incisions, punctures, lacerations, ulcers, sores, burns and the like to aid in stopping bleeding, and in preventing and treating wound contaminations.

In another method of use aspect, the present invention relates to oral applications of the dressing of the present invention, for treatment of oral mucosal surfaces, including wounds.

In yet another method of use aspect, the present invention relates to treatment of internal tissues, including nasal, aural, rectal, peritoneal, or vaginal surfaces.

In yet another method of use aspect, the present invention relates to applying the dressing of the present invention to a wound.

Yet another object of the present invention is to provide a dressing (preferably comprised of HYPOL polyurethane) with a soft and flexible structure that is easy to handle and apply to a wound surface, without risking contamination of the dressing and/or wound site; is minimally adherent to reduce wound pain and to minimize or avoid re-injuring the wound site upon removal; and, provides a cooling effect upon application.

Another object of the invention is to provide a method for making the non-medicated multi-layered polyurethane cooling dressing of the present invention.

In a method of use aspect, the present invention relates to a method of therapeutically or cosmetically treating the skin by application of the dressing of the present invention.

Upon study of the specification, the accompanying drawings and the appended claims, other objects and advantages of the present invention will become apparent to those skilled in the art. It is intended to cover all alternatives, modifications and equivalents as may be included within the spirit and scope of the invention as defined by the appended claims. As an example, while the present invention will be described herein as primarily useful as a surgical or first-aid wound dressing, it should be appreciated that a foam composition with similar characteristics could also be used for cosmetic, dental, biomedical, and other applications as well.

Having thus generally described the invention, reference will now be made to the accompanying drawings.

### BRIEF DESCRIPTION OF THE SEVERAL VIEWS OF THE DRAWINGS

Certain embodiments of the invention are described by way of illustration, without limitation of the invention thereto, other than as set forth in the present claims, with reference being made to the accompanying drawings, wherein:

FIG. 1A is a perspective view of a first embodiment dressing in accordance with the present invention, having two polyurethane layers;

FIG. 1B is a cross-sectional view of a portion of the FIG. 1A embodiment;

FIG. 2A is a perspective view of a second embodiment dressing in accordance with the present invention, having three polyurethane layers;

FIG. 2B is a cross-sectional view of a portion of the FIG. 2A embodiment;

FIG. 3A is a perspective view of a third embodiment dressing in accordance with the present invention, having three polyurethane layers and an elastomeric attachment layer;

FIG. 3B is a cross-sectional view of a portion of the FIG. 3A embodiment;

FIG. 4 is a graphical representation comparing the cooling efficacy of the subject dressing applied on intact human skin to that of commercially available dressings;

FIG. 5 is a graphical representation comparing the cooling efficacy of the subject dressing applied on rat burn wounds to that of commercially available dressings.

### DETAILED DESCRIPTION OF THE INVENTION

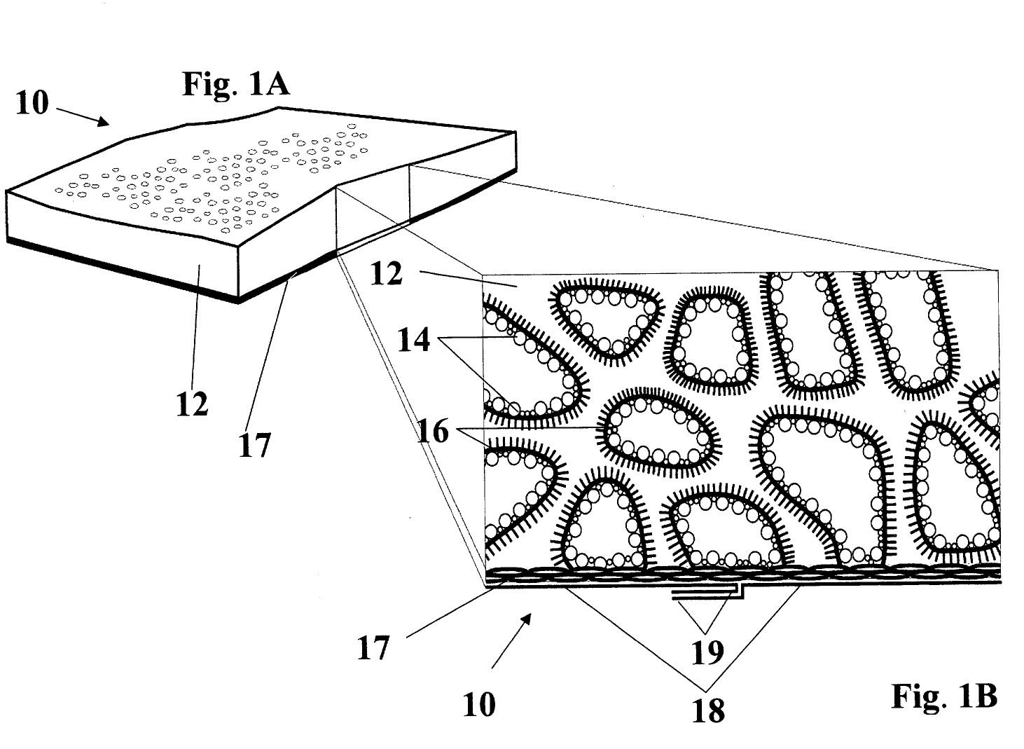

Referring to FIGS. 1A and 1B, a dressing 10, being a first embodiment of the present invention, has a layer 12 of polyurethane foam, preferably the aforementioned HYPOL. The layer 12 may constitute a reservoir for a selected drug or other therapeutic agent, represented by the elements 14 and 16. The layer 12 is adhered to a hydrogel polymer layer 17, which may contain at least one drug (not shown). The outer (lower) face of the layer 17 is protected by a two-piece cover-sheet 18, well known in the art, and having a pair of pull-tabs 19, to facilitate removal of the cover-sheet 18. In use, the protective cover-sheet 18 is removed from the dressing 10, and the outer hydrogel layer 17 is applied to the injured surface. The cover-sheet 18 preserves the sterility of the subject invention, and sustains the hydration of the hydrogel layer 17. Used as a surface dressing, the dressing 10 may be secured in place by way of a secondary dressing such as a bandage, tubular dressing, etc. (not shown). The form of the dressing 10 may be of cylindrical shape, for use as a packing within a deep wound, or a body cavity, where a fastening means is not usually required. The first embodiment of the subject dressing may also be used as a minimally adherent containment device for spilled internal organs.

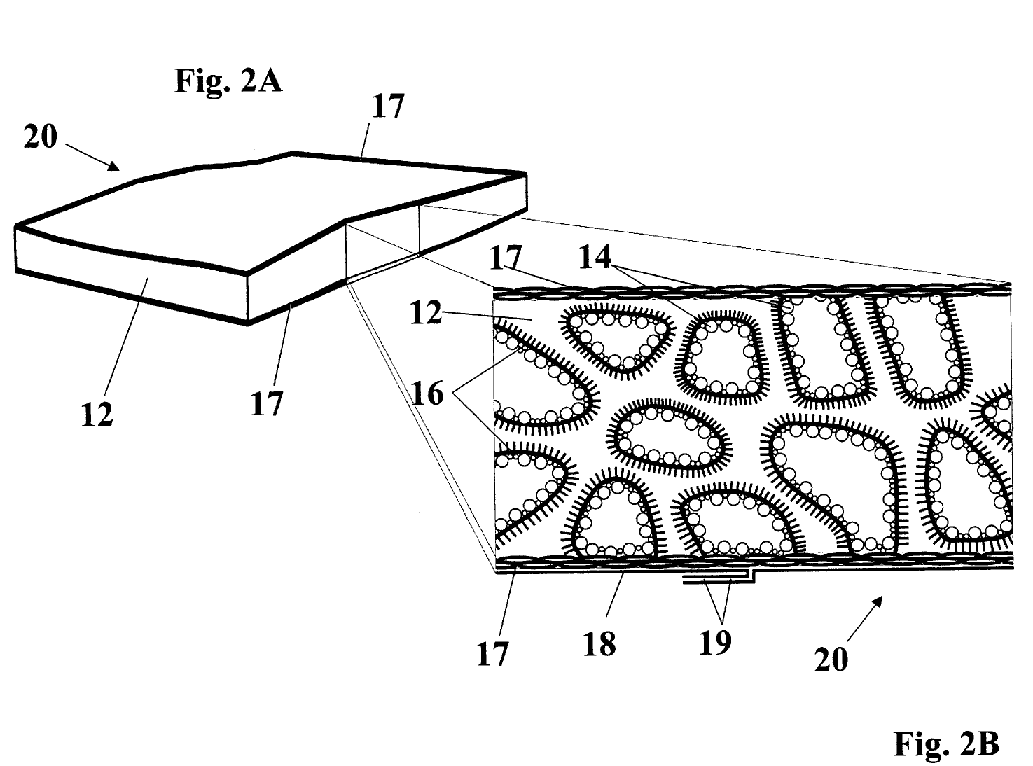

Turning to the FIG. 2 embodiment, a dressing 20, being a second embodiment of the present invention, has a fluid-retaining layer 12 comprised of a hydrophilic polyurethane foam layer, being illustrated as having two drugs 14, 16 in dispersed relation therein; the layer 12 being cast, as disclosed herein, over a surface-contacting hydrogel polymer layer 17, which may contain at least one drug therein (not shown), and is protected by a cover sheet 18. A second hydrogel polymer layer 17, which also may contain at least one drug therein, is cast in adhering relation on top of the drug reservoir layer 12. This top layer 17 also may be protected by a cover-sheet 18 (not shown). In use, the second embodiment multi-layered dressing 20 (after removal of both portions of the cover-sheet 18) may be used as a packing material for internal wounds, such as peritoneal wounds or nasal wounds, and thus does not require further means of attachment to the patient other than what is dictated by conventional surgical procedures.

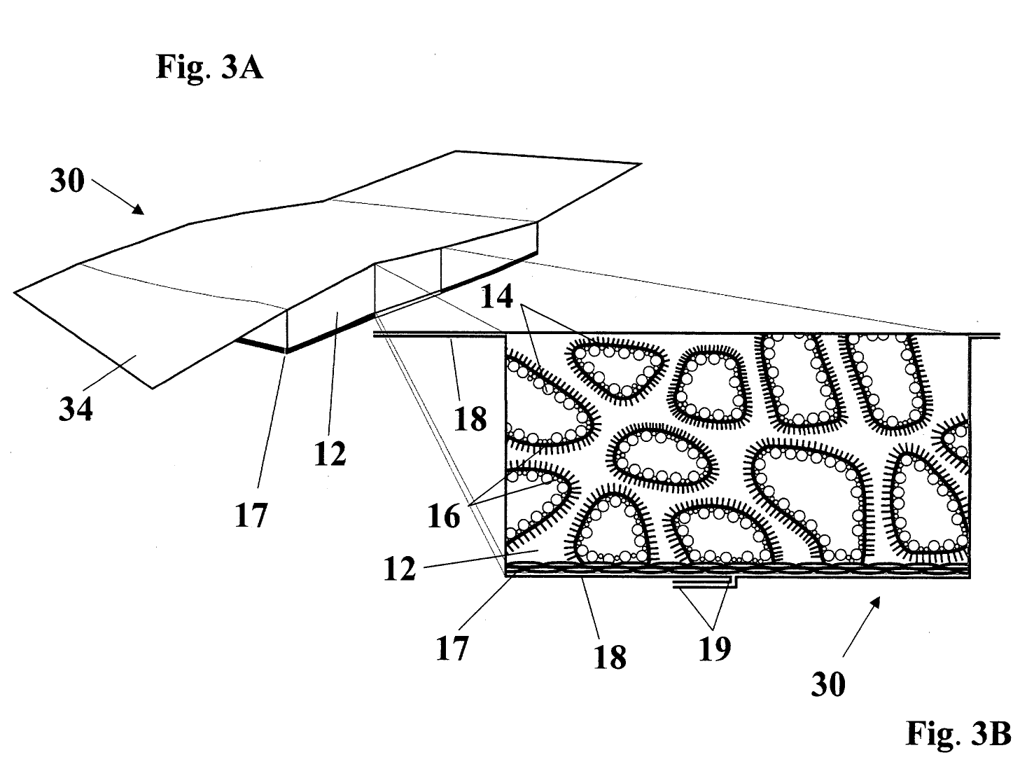

Referring to FIG. 3, in this third embodiment of the invention, the bi-layer dressing 30 has a fluid-retaining layer 12 comprised of a hydrophilic polyurethane foam layer being illustrated as having two drugs 14, 16 in dispersed relation therein. This layer 12 is cast as disclosed herein over a wound surface-contacting layer 17 comprised of a hydrogel polymer, the layer 17 also containing at least one drug (not shown). An adhesive outer elastomeric layer 34, extending beyond the edges of the layer 12 and providing a means of attachment to the patient, completes the dressing 30 as a bi-layer wound dressing. The two adhesive under-surfaces of the layer 34 and the hydrogel layer 17 are each protected by a respective cover-sheet 18. In use, the third embodiment of a multi-layered drug delivery device 30 can be used as a band-aid for superficial wounds or as a compression bandage for hemorrhagic penetrating wounds.

The elastomeric adhesive outer layer 34, is selected from a group of materials including, but not limited to polyurethane, polyethylene, vinyl, polyvinylchloride, or other suitable material. The elastomeric outer layer 34 is water-vapour and gas-permeable; substantially liquid-impermeable to prevent leakage of wound exudates from the dressing; and is also microbe-impermeable. The perimeter portion of the elastomeric adhesive outer layer 34 is coated with a suitable medical-grade adhesive, and is preferably perforated to allow the skin of the patient to breathe.

Alternatively, the outer layer 34 may be bonded to the foam layer 12 using conventional bonding methods (e.g., heat). This elastomeric adhesive outer layer 34 serves to secure the dressing to the patient and/or to apply adequate pressure to stop bleeding. It will be understood that the reactant mixture formulation of the HYPOL foam layer can be readily tailored to provide for application either as a ‘band-aid’ for superficial wounds (including scrapes, abrasions, donor sites), or as a compression bandage for hemorrhagic penetrating wounds.

The third embodiment 30 does not require the use of a secondary dressing, but involves merely the removal of the cover sheet 18 prior to its application to the wound.

Concerning the make-up and fabrication of the subject dressings, they can be manufactured to assume various shapes (cylindrical, oval, tubular, etc.) or flat sheets of various predetermined sizes. Preferably, the dressings are prepared under aseptic conditions, packaged in aluminum foil laminated bags with a heat sealable film, and sterilized in the package. Favoured procedure is by gamma-sterilization. Alternatively, the dressing can be sterilized by ethylene oxide and heat sterilization.

The preferred composition of each of the layers of the dressings of the present invention are described in detail herein. When used herein the term hydrophilic polymer foam means any foam that will absorb fluids such as water, blood, wound exudates (including blister fluid) and other body fluids (including peritoneal fluid).

Appropriate hydrophilic foams include polyurethane; carboxylated butadiene-styrene rubber; polyacrylate; polyvinylic; polyester or cellulosic foams; polyurethane prepolymers derived from methylenediphenyl diisocyanate (MDI) or tolylene diisocyanate (TDI) prepolymers; or hydrophilic epoxy foams. Polyvinylic foams include polyvinyl acetal foams formed by the reaction of polyvinyl alcohol and an aldehyde.

Favoured hydrophilic polymer foams are hydrophilic polyurethane foams, especially cross-linked polyurethane foams. HYPOL pre-polymer foams form a preferred group of foams within the general description of hydrophilic polymer foams. HYPOL foams can be made from HYPOL hydrophilic isocyanate terminated pre-polymers marketed by DOW Chemicals Inc. Ideally, the hydrophilic foam will absorb at least four times its weight in fluids. Suitable foams may be prepared from hydrophilic materials per se or may be treated to render them hydrophilic (e.g., with surfactants, superabsorbent agents, etc.). However, it is preferable that the foam be highly hydrophilic per se, since the incorporation of chemicals (including surfactants, etc.) may alter the physical characteristics (e.g., absorption, porosity, pore size, etc.) of the foam material. It is also desirable that the hydrophilic polymer foam layer absorbs the wound exudate rapidly as this prevents undesirable maceration of the wound by the accumulation of exudates underneath the dressing. The hydrophilic foam used should also be conformable (i.e., soft and compressible, not stiff or rigid). That is, the hydrophilic foam, when placed in a body cavity, will conform readily to the contours of the wounds, whether the patient is resting or moving.

The hydrophilic polyurethane foam may be comprised of a single type of polymer, although blends may be used to form the hydrophilic foam. For example, polyurethane foam polymerized with polyether-polyamides; polyether polyurethane; polyvinyl alcohols; and mixtures thereof

The polyurethane hydrophilic foam may be cross-linked with chitin; collagen; fibrin; aliginate; glycosaminoglycan; polyvinyl-lactam such as polyvinylpyrrolidone, polyvinylbutirolactam, polyvinylcaprolactam and the like; cellulose derivatives; benzene-1,2,4-tricarboxylic acid; nitrilotriacetic acid; citric acid; 4,4-methylenebis(o-chloroaniline); or mixtures thereof.

Suitable polyols may be mixed with the polyurethane hydrophilic foam, and include, but are not restricted to, water soluble alcohols, including monols, diols and polyhydric alcohols. Examples of monols include ethyl alcohol and isopropyl alcohol. Exemplary of suitable diols are propylene glycol, polyethylene glycol and polypropylene glycol. Exemplary of suitable polyhydric alcohols are glycerin, 1,2,4-butanetriol, trimethylolpropane, pentaerythritol and sorbitol.

Suitable catalysts may also be added to the polyurethane hydrophilic foam to produce desirable physical characteristics. Such catalysts include, but are not restricted to, dimethylethanolamine; diethylenetriamine; triethylenetetramine; tetraethylenepentamine; polyethyleeneimine; glycerol; trimethylolpropane; pentaerythritol; tolylene-2,4,6-triamine; ethylene diamine; amino-ethanol; trimethylenediamine; tetramethylenediamine; pentamethylene-diamine; hexamethylene-diamine; ethanolamine; diethanolamine; hydrazine; triethanolamine; n-methyl morpholine; n-ethyl morpholine; trimethylamine; triethylamine; tetramethyl butane diamine; triethylene diamine; dimethylaminoethanol; benzyldimethylamine; dibutyl tim dilaureate; and stannous octoate.

Useful additives may also be added to the polyurethane foam, and include, but are not limited to, organic and inorganic salts; alcohols; amines; acids; polymer latices; resin or wax dispersions; fillers; fibers; cellulosics; surfactants; pigments; dyes; enzymes; proteins; chelates; thickeners; stabilizers; and so forth.

The foam layer may contain a number of other chemicals described in detail herein, to further improve its hydrophilic properties. Surfactants may be included in this layer. Suitable and preferred biocompatible surfactants forming conformable hydrophilic polymer foams include, but are not limited to, non-ionic surfactants, such as oxypropylene-oxyethylene block co-polymers known as Pluronics™ marketed by BASF Wyandotte, preferably Pluronic F68 sorbitan trioleate; polyoxyethylene sorbitol oleate; polyoxyethylene sorbitan monolaureate; polyoxyethylene lauryl ether; polyoxyethylene stearyl ether; fluorochemical surfactants; and block copolymer condensates of ethylene oxide and propylene oxide with propylene glycol; methylcellulose; guar gum; pectin; karaya gum; agar; acacia powder; gelatin; and other hydrophilic polymers and combinations thereof. Generally but not necessarily, the amount of surfactant should be up to 10% by weight of the foam reactant mixture. The selected surfactant should not react with the selected pre-polymer, or with any component of the reactant mixture as to impair foam formation or to adversely affect the desired characteristics of the foam composition, in use or while being stored.

Other potential additives could include chitosan, aliginate, etc., to improve the hydrophilic action of the HYPOL pre-polymer. Thus, the foam reactant composition may include a hydrophilic agent that is incorporated into the foam mixture to absorb liquid (e.g. wound exudate, peritoneal fluid). The hydrophilic agent is preferably a highly absorbent polymer, commonly known as a superabsorbent polymer. Inclusion of such agent will increase the capacity of the wound dressing to retain at least twice its weight in fluid after compression. The amount and type of hydrophilic agent used in the wound dressing will be governed by the intended application of the invention. For example, for an ulcerating wound with large fluid exudate volume (e.g., a burn or a bleeding wound), a hydrophilic agent with a high uptake is desirable. On the other hand, for a laceration or abrasion, it may be more suitable to use a less hydrophilic agent or to use an agent with a lower fluid uptake.

Determination of the types and amounts of adjuvant, surfactant and hydrophilic agent used is well within the ability and knowledge of one skilled in the art, in light of the disclosure contained herein.

The surface-contacting hydrogel layer is preferably made of HYPOL, but may be made of any of the known hydrogels heretofore employed for wound treatment and already known to those skilled in the art. The hydrogel may be comprised of, or cross-linked with, polyvinyl alcohol; polyvinylpirrolydone; polyvinyl-lactam; collagen; dextran; cellulose; hyaluronate; chitin; chitosan; agar; agarose; alginate; carrageenan; silicone; polyurethane; polyethylene oxide-based diamine; gelatin; glycerine; polyoxamides; polyesters; polyoxaesters; vinyl esters polymers; vinyl carboxylic acids and salts; polyacrylamide; dimethylacrylamine co-polymers; or compatible mixtures thereof.

The surface-contacting hydrogel layer may also comprise cryogels or other gels produced without the use of cross-linking agents or other adjuvants.

The surface-contacting hydrogel layer may further include hydrogel-forming agents. For example, vinyl-cross-linked polyethylene oxides; polyurethane ureas; polysiloxanes; mixtures of gellable polysaccharides such as carboxymethyl cellulose, carboxymethyl starch and hydroxypropyl cellulose; proteins; hydrophilic polymers; graft polymers of hydrolyzed starch; polyacrylonitrile; nonionic agents such as polyvinyl alcohol, and polyvinyl ethers; cationic agents such as polyvinyl pyridine, polyvinyl morpholinione; and combinations thereof.

The surface-contacting hydrogel layer may further include a number of other chemicals as described herein, to further improve its cooling characteristics. For example, polypropylene glycol; propylene glycol; polyethylene glycol; dipropylene glycol; and the like.

The type and amount of pre-polymer in the reactant mixture used to prepare each of the layers will depend on a number of factors, including the proportion of other components in the reactant mixture. In all instances, there should be sufficient pre-polymer and water to form a polyurethane foam or hydrogel layer of suitable thickness. There should also be sufficient pre-polymer to provide integrity to each of the layer compositions, but not so much that the resulting compositions become unworkable.

The reactant mixtures of each layer of the subject dressings may further include an adjuvant to extend the curing time of the foam or hydrogel reactant mixture, thereby allowing a thorough mixing of the mixtures prior to spreading them sequentially into layers of suitable thickness, for curing. Preferably, the adjuvant selected is water soluble and biocompatible (i.e., does not exert harmful effects upon contacting the wound bed or skin). It is also preferable that the selected adjuvant be compatible with the pre-polymers selected as well as with the therapeutic agents or other additives incorporated into the reactant mixtures, Suitable adjuvants include water soluble alcohols, including monols, diols, and polyhydric alcohols.

Preferably, the reactant foam or hydrogel mixtures should contain less than 0.01% of alcohol by weight.

A first method of manufacture of the subject dressing consists of the steps of mixing the appropriate reactants of the surface-contacting hydrogel layer together in an appropriate receptacle to form a standardized aerated mix. The mixture is then spread at room temperature onto a smooth support to which it is not adherent (e.g., a glass surface) to form a layer of pre-determined thickness. The spreading may be effected by means of a spreader bar that is drawn over the surface of the mix at a fixed distance above the support surface. The second layer (i.e., the hydrophilic foam layer) is simultaneously prepared in the same manner, and applied over the wound-contacting layer before it is totally cured, in order to achieve adhesion between the layers.

In a further method of manufacture of a dressing of the present invention, a third layer comprising HYPOL hydrogel would be immediately cast on top of the hydrophilic polymer foam layer.

For another embodiment, the mixture of the surface-contacting hydrogel layer is spread as described herein over a fully cured hydrophilic foam layer.

For yet another embodiment, a third layer comprising HYPOL hydrogel is cast on top of the hydrophilic foam layer already in adhering relation to another surface-contacting hydrogel layer.

In yet another method of manufacture, all layers are prepared, cast and spread individually, and then sealed together using known methods of lamination (e.g., heat sealing, radio frequency welding, discontinuous adhesive, ultrasonic welding). It is further desirable that the pre-polymers selected are preferably capable of curing in the absence of catalysts, and at ambient temperature.

After preparing the dressing embodiments of the present invention as described herein, the surface-contacting hydrogel layer may be perforated or sliced through its thickness in several sites to create channels to enhance absorption of exudates.

For yet another embodiment, the mixture of the surface-contacting hydrogel layer is sprayed over a fully cured hydrophilic foam layer to form a discontinuous hydrogel layer, thus enhancing absorption of exudates into the hydrophilic polyurethane foam layer.

After curing, the surface-contacting hydrogel layer will generally although not necessarily have a thickness of up to 2.54 mm, and preferably in the range 0.76 to 1.27 mm. After curing, the hydrophilic polymer foam layer will generally, although not necessarily, have a thickness of up to 10 mm, and preferably in the range of 3 to 7 mm. It will be appreciated by those skilled in the art that the thickness of the layers will depend, however, on a variety of considerations, including the quantity of each additive to be incorporated in each of the layers, the level of absorbency or cooling required, and the like.

The following examples show by way of illustration and not by way of limitation, the practice of the present invention.

### EXAMPLE 1

This first example describes how experimental dressings of the present invention were prepared under laboratory conditions. The two layers that comprised the dressing were prepared separately. Briefly, the hydrogel layer was first cast onto a glass plate and within approximately 2 minutes, the foam-based second mixture was poured over it. The resulting dressings consisted of a hydrogel layer with a thickness of about 0.25 mm (10 mils) and a foam layer with a thickness of about 3.2 mm (128 mil). Process parameters (e.g., reaction temperatures, mixing speeds and mixing times) were determined in preliminary experiments and selected to optimize the time available for preparing the specimen, before increasing viscosity of the solution interfered with the process.

The hydrogel layer was prepared as follows. Water was added to a mixture consisting of the pre-polymers Hypol 2060G and Hypol G50 (72%±0.4 w/w) at a ratio of 5:1. The aqueous dispersion was mixed at 800±100 rpm for 30 seconds. The solution was poured onto a glass plate (30 cm×30 cm) between two borders (10 cm apart). The borders were made of a ultra high molecular weight polyethylene (UHMWPE) tape (10 mils thick) with an acrylic adhesive backing. The aqueous mixture was then spread between the borders using a glass plate covered with a 3 mils thick silicone adhesive polytetrafluoroethylene (PTFE) tape. The solution was spread in one direction followed by a second spreading pass in the opposite direction. The hydrogel layer was allowed to set for 2 minutes prior to pouring the foam layer over it. The hydrogel layer needed to have almost set-up prior to pouring the second, foam-based solution over it; otherwise a non-set hydrogel could be displaced by the second solution, and a non-uniform hydrogel layer would result.

The foam layer was prepared during the casting of the hydrogel layer. Chilled water was added to pre-weighed Hypol 2002 pre-polymer at a ratio of approximately 1.5:1, and the solution was mixed at 3000±200 rpm for 30 seconds. After mixing, the solution was poured over the hydrogel layer between two silicone rubber borders (3.2 mm thick) placed on top of the UHMWPE tape borders used in casting the hydrogel layer. The foam solution was spread slowly in one direction followed by a second spreader pass in the opposite direction.

After setting for 10 minutes, a razor knife was drawn along the interior edge of the borders to facilitate removal of the sample. Typically, 2-4 cm off each end of the sample was discarded by trimming across the sample with a blade. The dressing was readily peeled from the glass plate. Sample portions were then placed in sterile aluminum pouches and heat-sealed until they were used.

### EXAMPLE 2

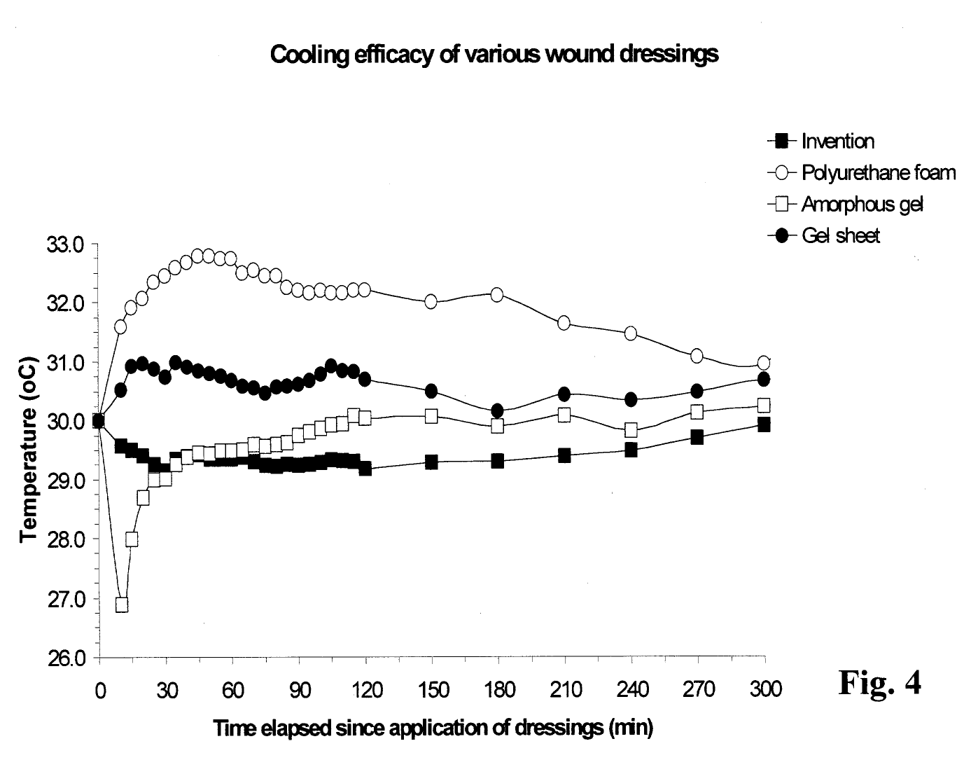

### Test Study (Reference FIG. 4)

The objective of this study was to compare the effectiveness of various non-medicated dressings in cooling intact human skin. On the day of the experiment, the skin over the triceps of both arms of eight subjects was cleansed using alcohol swabs. Two small thermistors were taped 5 cm apart on the skin of each arm, the probes being positioned approximately 10 cm from the tip of the shoulder. The four experimental groups of dressings tested were: a non-medicated dressing of the present invention as well as three commercially available wound-care products comprising: a hydrogel sheet, a polyurethane foam dressing, and an amorphous gel dressing.

An experimental dressing was applied to each arm of each subject, being respectively centered over the two thermistors, and covered with a tape. Each dressing was then further secured in place using a 15 cm wide self-adherent non-woven wrap. Temperature recordings from the thermistors were acquired for 6 hours using a small data logger worn on a belt.

The effectiveness of the various dressings in cooling the skin is shown in FIG. 4. Within 30 min after application of the gel sheet and the polyurethane foam dressing, the skin temperature of the upper arm (Tₛₖᵢₙ) had risen by 1.0° C. and 2.5° C., respectively. While Tₛₖᵢₙ remained constant (30.8° C.) for the remainder of the study in the gel-sheet group, it further increased in the polyurethane-foam group, reaching a maximum of 33° C. after 60 min. Tₛₖᵢₙ slowly declined for the remainder of the 6-h study, remaining above Tₛₖᵢₙ recorded prior to application of the dressing. In contrast, Tₛₖᵢₙ markedly dropped (3.0° C.) within 10 min of applying the amorphous gel, while Tₛₖᵢₙ under the dressing of the present invention dropped by 1.0° C. However, the greater cooling effect of the gel dressing was short-lived, Tₛₖᵢₙ after 30 min being comparable to that observed for the dressing of the present invention. While Tₛₖᵢₙ remained constant (29.2° C.) under the dressing of the present invention for most of the 6-hour study, it increased steadily under the amorphous gel, reaching a plateau of 30° C. after 90 min. These data demonstrate that the dressing of the present invention can offer a sustained cooling effect for at least 6 hours.

### EXAMPLE 3

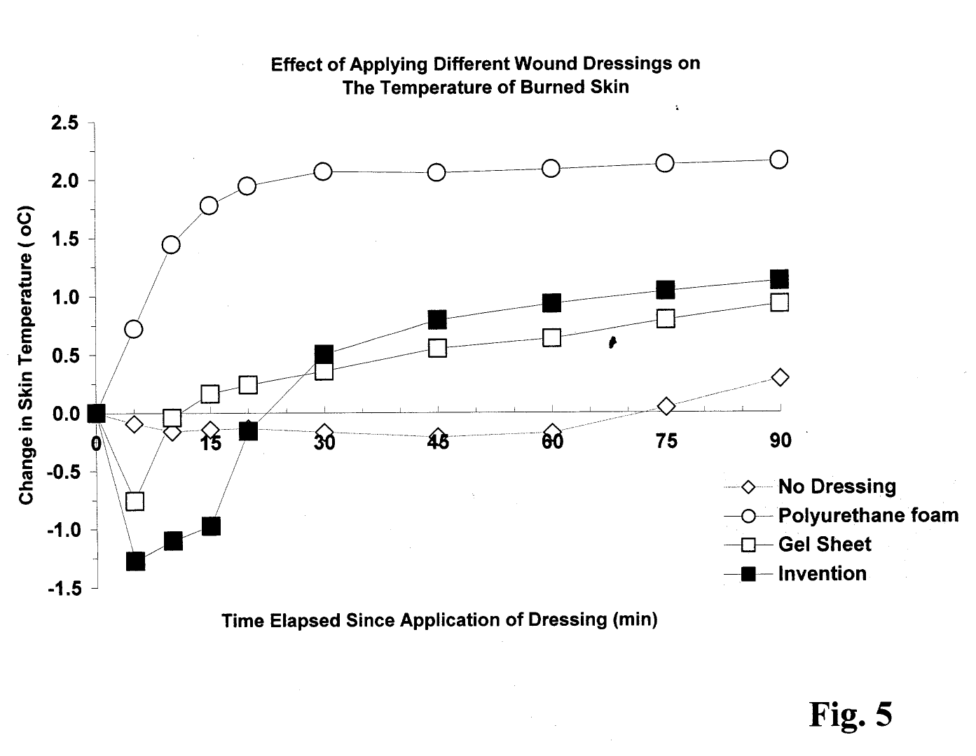

### Test Study (Reference FIG. 5)

The objective of this study was to compare the effectiveness of various non-medicated dressings in cooling a burn wound. Rats were anesthetized, and a 30% full-thickness scald wound was made on their dorsum. Three small thermistors were taped 1.5 cm apart on the dorsal skin, the first probe being positioned approximately 2 cm from the nape of the neck. The three experimental groups of dressings tested were: a non-medicated dressing of the present invention as well as two commercially available wound-care products comprising: a hydrogel sheet and a polyurethane foam dressing. A control group of animals was included to measure the temperature of the skin when no dressing was applied other than the securing wrap. The foam layer of the wound dressing of the present invention was wetted prior to its application to the burn wound with a volume of warm saline corresponding to 50% of the dressing's maximal absorption capacity, to provide an estimate of the cooling efficacy of a ‘wet dressing’ under a simulated a condition of moderate to high level of wound exudates.

An experimental dressing was applied to cover the three thermistors. Each dressing was then further secured in place using a 10 cm wide self-adherent non-woven wrap, and the animals were returned to their cages. Temperature recordings from the thermistors were acquired for 90 min using a small data logger attached to the lid of the cage.

The changes in skin temperature following application of the various dressings on the burn wound is shown in FIG. 5. There was no significant reduction in T_{burn} when no dressing was applied other than the securing wrap. In contrast, the skin temperature of the burn wound (T_{burn}) had risen by 0.8° C. within 5 min after application of the polyurethane foam dressing. T_{burn} further increased for the next 30 min, stabilizing for the remainder of the study 2° C. above the T_{burn} recorded for the control animals. Application of either the gel sheet or the dressing of the present invention exerted a cooling effect on the burn wound. However, the reduction in T_{burn} was 90% greater within the first 5 min following application of the present invention than that of the gel sheet. Furthermore, while the cooling effect of the gel sheet was observed only for 5 min, T_{burn} remained below that of the control burn wounds for up to 20 min. These data demonstrate that the efficiency of the dressing of the present invention to cool burn wounds is greater than that of commercially available hydrogel dressings.

The foregoing experimental determination of the cooling efficacy of the subject dressing should be readily validated and replicated by those skilled in the art.

The dressing examples and methods described and disclosed herein are intended to be illustrative and not exhaustive. These examples and descriptions will suggest many variations and alternatives to one of ordinary skill in the art. All of these alternatives and variations are encompassed within the scope of the following claims.

1. A dressing comprising:
a) a hydrophilic polymer foam layer serving in use as a fluid-retaining reservoir, and
b) a hydrogel surface-contacting layer, serving in use as a surface cooling and anti-adhesion agent

2. The dressing as set forth in claim 1, said surface-contacting layer consisting of a hydrogel incorporating or cross-linked with an element selected from the group consisting of polyvinyl alcohol; polyvinylpirrolydone; polyvinyl-lactam; collagen; dextran; cellulose; hyaluronate; chitin; chitosan; agar; agarose; alginate; carrageenan; silicone; polyurethane; polypropylene glycol; propylene glycol; polyethylene glycol; polyethylene oxide-based diamine; gelatin; glycerine; polyoxamides; polyesters; polyoxaesters; vinyl esters polymers; vinyl carboxylic acids and salts; polyacrylamide; dimethylacrylamine co-polymers; and combinations thereof.

3. The dressing as set forth in claim 1, wherein said surface-contacting layer consists of a hydrogel incorporating an element selected from the group consisting of vinyl-cross-linked polyethylene oxides; polyurethane ureas; polysiloxanes; mixtures of gellable polysaccharides including carboxymethylcellulose, carboxymethyl starch and hydroxypropyl cellulose; proteins; hydrophilic polymers; graft polymers of hydrolyzed starch; polyacrylonitrile; nonionic agents including polyvinyl alcohol, and polyvinyl ethers; cationic agents including polyvinyl pyridine, polyvinyl morpholinione; and combinations thereof.

4. The dressing of claim 1 wherein said hydrophilic polymer foam layer is comprised of a hydrophilic foam.

5. The dressing of claim 4 wherein said hydrophilic foam is selected from the group consisting of polyurethane; carboxylated butadiene-styrene rubber; polyacrylate; polyvinylic; polyester foams; cellulosic foams; polyurethane pre-polymers derived from the group consisting of methylenediphenyl diisocyanate (MDI) and tolylene diisocyanate (TDI) pre-polymers; hydrophilic epoxy foams; polyvinylic foams; polyvinyl acetal foams formed by the reaction of polyvinyl alcohol and an aldehyde; hydrophilic polymer foams; hydrophilic polyurethane foams; cross-linked polyurethane foams; HYPOL pre-polymer foams; and combinations thereof.

6. The dressing of claim 4 wherein said hydrophilic foam is cross-linked with a substance selected from the group consisting of chitin; collagen; fibrin; aliginate; glycosaminoglycan; polyvinyl-lactam such as polyvinylpyrrolidone, polyvinylbutirolactam, polyvinylcaprolactam and the like; cellulose derivatives; benzene-1,2,4-tricarboxylic acid; nitrilotriacetic acid; citric acid; 4,4-methylenebis(o-chloroaniline); and mixtures thereof.

7. The dressing of claim 4 wherein said hydrophilic foam is mixed with a polyol.

8. The dressing of claim 7, wherein said polyol is selected from the group consisting of water soluble alcohols, monols, diols and polyhydric alcohols, ethyl alcohol, isopropyl alcohol, propylene glycol, polyethylene glycol, polypropylene glycol, propylene glycol, dipropylene glycol, glycerin, 1,2,4-butanetriol, trimethylolpropane, pentaerythritol, sorbitol, and other polyols and compatible mixtures thereof.

9. The dressing of claim 4, wherein said hydrophilic foam is mixed with a surfactant.

10. The dressing of claim 9, wherein said surfactant is selected from the group consisting of sorbitan trioleate; polyoxyethylene sorbitol oleate; polyoxyethylene sorbitan monolaureate; polyoxyethylene lauryl ether; polyoxyethylene stearyl ether; fluorochemical surfactants; and block copolymer condensates of ethylene oxide and propylene oxide with propylene glycol; methylcellulose; guar gum; pectin; karaya gum; agar; acacia powder; gelatin; and other surfactants and combinations thereof.

11. The dressing of claim 4, wherein said hydrophilic foam is mixed with a catalyst.

12. The dressing of claim 11, wherein said catalyst is selected from the group consisting of dimethylethanolamine; diethylenetriamine; triethylenetetramine; tetraethylenepentamine; polyethyleeneimine; glycerol; trimethylolpropane; pentaerythritol; tolylene-2,4,6-triamine; ethylene diamine; amino-ethanol; trimethylenediamine; tetramethylenediamine; pentamethylene-diamine; hexamethylene-diamine; ethanolamine; diethanolamine; hydrazine; triethanolamine; n-methyl morpholine; n-ethyl morpholine; trimethylamine; triethylamine; tetramethyl butane diamine; triethylene diamine; dimethylaminoethanol; benzyldimethylamine; dibutyl tim dilaureate; and stannous octoate.

13. The dressing of claim 4, wherein said hydrophilic foam is mixed with an additive selected from the group consisting of organic and inorganic salts; alcohols; amines; acids; polymer latices; resin dispersions; wax dispersions; fillers; fibers; cellulosics; surfactants; pigments; dyes; enzymes; proteins; chelates; aliginates; chitosan; superabsorbent agents; thickeners; and, stabilizers.

14. The dressing of claim 1 which further comprises an outer compatible elastomeric layer, wherein said outer layer is positioned on the side of the dressing opposite from the side which comes into contact with the tissue site.

15. The dressing as set forth in claim 14, wherein said outer layer comprises an elastomeric material selected from the group consisting of polyurethane, polyethylene, vinyl, and polyvinylchloride.

16. The dressing of claim 1 which further comprises a protective release sheet positioned in adhering, protective relation with said hydrogel surface-contacting layer.

17. The dressing of claim 1 wherein said surface-contacting layer has a plurality of channels within said layer to promote dispersion of fluid to said hydrophilic polyurethane foam layer.

18. The dressing of claim 17 characterized in that said layer consists of a plurality of separate layer portions in mutually spaced, sprayed adhering relation to said hydrophilic foam layer.

19. The dressing of claim 1 which further comprises a second hydrogel surface-contacting layer in adjoining relation to said hydrophilic foam layer.

20. The dressing of claim 18 wherein said surface-contacting layer is discontinuous, to provide fluid distribution channels.

21. The dressing of claim 1, including flow sensitive transfer means located in interposed relation between said hydrophilic layer and said hydrogel layer, to control the transfer of fluid between said layers, whereby deformation of a said layer is substantially controlled.

22. The dressing of claim 1, wherein said polyurethane hydrogel is a hydrophilic polyurethane prepolymer.

23. The dressing of claim 1, wherein said device has a cooling effect on a surface when in direct contact with said surface.

24. The dressing of claim 1, wherein said device is substantially non-adherent when applied to the person of a patient.

25. The dressing of claim 1, wherein, in use, said hydrophilic polyurethane foam layer is absorbent of body fluids.

26. A method for manufacturing the dressing of claim 1, said method comprising:
a) mixing the components of said surface-contacting layer together to form a standardized aerated mixture;
b) spreading said aerated mixture onto a smooth support to form a layer of pre-determined thickness;
c) mixing the components of said hydrophilic foam layer together to form a standardized aerated mixture; and
d) spreading said aerated mixture of step c) on top of said mixture of step b) and allowing both layers to cure, whereby mutual adherence occurs.

27. The method as set forth in claim 26 wherein said step d) is carried out before the mixture of step b) has fully cured.

28. The method as set forth in claim 26 wherein said step d) is carried out after the mixture of step b) has fully cured.

29. A method for manufacturing the dressing of claim 1, said method comprising:
a) mixing the components of said hydrophilic foam layer together to form a standardized aerated mixture;
b) spreading said aerated mixture onto a smooth support to form a layer of pre-determined thickness;
e) mixing the components of said surface-contacting hydrogel layer together to form a standardized aerated mixture; and
f) spreading said aerated mixture of step c) on top of said mixture of step b) and allowing both layers to cure, whereby mutual adherence occurs.

30. The method as set forth in claim 29 wherein said step d) is carried out before the mixture of step b) has fully cured.

31. The method as set forth in claim 29 wherein said step d) is carried out after the mixture of step b) has fully cured.

32. The method of claim 26, wherein said method further comprises mixing the components of a second surface-contacting layer together to form a standardized mixture and spreading said mixture as a layer of predetermined thickness on top of the mixture of step c).

33. The method of claim 29, wherein said method further comprises mixing the components of a second surface-contacting layer together to form a standardized mixture and spreading said mixture as a layer of predetermined thickness on top of the mixture of step b).

34. A method for manufacturing the dressing of claim 1, said method comprising:
a) mixing the components of said surface-contacting layer together to form a standardized mixture;
b) spreading the mixture onto a smooth support to form a layer of pre-determined thickness, and allowing the layer to fully cure;
c) mixing the components of said hydrophilic polyurethane prepolymer foam layer together to form a standardized mixture;
d) spreading the mixture of step c) onto a smooth support to form a layer of pre-determined thickness, and allowing the sheet to fully cure; and
g) sealing the layer obtained in step b) to the layer obtained in step d).

35. A method for manufacturing the dressing of claim 1, said method comprising:
a) mixing the components of said hydrophilic polyurethane foam layer together to form a standardized mixture;
b) spreading the mixture onto a smooth support to form a layer of pre-determined thickness;
c) mixing the components of said surface-contacting layer together to form a standardized mixture; and
d) spreading the mixture of step c) on top of the mixture of step b) once the mixture of step b) has fully cured and,
e) sealing the layer obtained in step b) to the layer obtained in step d).

36. The method of claim 34, wherein said layers are adjoined using a method selected from the group consisting of heat sealing, radio frequency welding, discontinuous adhesive and ultrasonic welding.

37. A method for cosmetically or therapeutically treating the skin of a patient, wherein said method comprises applying the dressing of claim 1 to the skin of said patient.

38. A method for therapeutically treating internally damaged tissue of a patient, wherein said method comprises applying the dressing of claim 1 to the internally damaged tissue of said patient.

39. The method of claim 38, wherein said internally damaged tissue is selected from the group consisting of oral, nasal, aural, rectal, peritoneal, and vaginal tissue.

40. A dressing comprising:
a) a cross-linked hydrophilic polyurethane foam layer, which comprises a cross-linked hydrophilic foam together with a polyol, a surfactant, and a further component selected from the group consisting of a catalyst and an additive; and
b) a polyurethane hydrogel surface-contacting layer.
